(19) United States
(12) Patent Application Publication
(10) Pub. No.: US 2021/0136257 A1
(43) Pub. Date: May 6, 2021

(54) ENDOSCOPE APPARATUS, OPERATING METHOD OF ENDOSCOPE APPARATUS, AND INFORMATION STORAGE MEDIUM

(71) Applicant: OLYMPUS CORPORATION, Tokyo (JP)

(72) Inventor: Naoya KURIYAMA, Tokyo (JP)

(73) Assignee: OLYMPUS CORPORATION, Tokyo (JP)

(21) Appl. No.: 17/145,475

(22) Filed: Jan. 11, 2021

Related U.S. Application Data
(63) Continuation of application No. PCT/JP2018/028891, filed on Aug. 1, 2018.

(51) Int. Cl. H04N 5/217; H04N 5/14; A61B 1/00; A61B 1/045

(52) U.S. Cl. CPC H04N 5/217; H04N 5/144; A61B 1/00009; A61B 1/045; A61B 1/00188

(57) ABSTRACT

An endoscope apparatus includes an imaging device that acquires a plurality of images with different focus positions at different timings and a processor including hardware. The processor aligns the plurality of images with different focus positions, combines the plurality of images with different focus positions that have been aligned into a single depth of field increased image to increase a depth of field, obtains a risk index indicating a risk of occurrence of artifact in the depth of field increased image, and corrects the depth of field increased image on a basis of the risk index.

### CROSS REFERENCE TO RELATED APPLICATION

This application is a continuation of International Patent Application No. PCT/JP2018/028891, having an international filing date of Aug. 1, 2018, which designated the United States, the entirety of which is incorporated herein by reference.

### BACKGROUND

An endoscope apparatus is required to have a depth of field as deep as possible so as not to pose a problem for diagnosis and treatment performed by a user. Unfortunately, the endoscope apparatus has recently included an image sensor having a large number of pixels and thus the depth of field has become shallower.

In order to compensate the shallow depth of field, introduction of an extended depth of field (EDOF) technology, which increases the depth of field, is proposed. For example, Japanese Translation of PCT International Application Publication No. JP-T-2013-513318 discloses a method for increasing the depth of field by capturing and aligning a plurality of images with different focus positions, and combining in-focus regions of the plurality of images.

### SUMMARY

In accordance with one of some aspect, there is provided an endoscope apparatus comprising: an imaging device that acquires a plurality of images with different focus positions at different timings; and a processor including hardware, the processor being configured to align the plurality of images with different focus positions, combine the plurality of images with different focus positions that have been aligned into a single depth of field increased image to increase a depth of field, obtain a risk index indicating a degree of a risk of occurrence of artifact in the depth of field increased image, and correct the depth of field increased image on a basis of the risk index.

In accordance with one of some aspect, there is provided an operating method of an endoscope apparatus comprising: acquiring a plurality of images with different focus positions at different timings; aligning the plurality of images with different focus positions; combining the plurality of images with different focus positions that have been aligned into a single depth of field increased image to increase a depth of field; obtaining a risk index indicating a degree of a risk of occurrence of artifact in the depth of field increased image, and correcting the depth of field increased image on a basis of the risk index.

In accordance with one of some aspect, there is provided a non-transitory information storage medium storing a program, the program causing a computer to perform steps of: acquiring a plurality of images with different focus positions at different timings; aligning the plurality of images with different focus positions; combining the plurality of images with different focus positions that have been aligned into a single depth of field increased image to increase a depth of field; obtaining a risk index indicating a degree of a risk of occurrence of artifact in the depth of field increased image, and correcting the depth of field increased image on a basis of the risk index.

### BRIEF DESCRIPTION OF THE DRAWINGS

FIG. 1 is a configuration example of an endoscope apparatus.

FIG. 2 is a configuration example of a risk index calculation section.

FIG. 3 is a graph illustrating a relational example between a motion amount and a risk index.

FIG. 4 is a flowchart illustrating a fast motion risk index calculation process.

FIG. 5 is a diagram illustrating a random motion risk index calculation process.

FIG. 6 is a flowchart illustrating the random motion risk index calculation process.

FIG. 7 is a flowchart illustrating a process of obtaining a peak value of evaluation values.

FIG. 8 is a flowchart illustrating a calculation process of a flat region risk index or a periodic structure risk index.

FIG. 9 is another configuration example of the risk index calculation section.

FIG. 10 is a graph illustrating a relationship between the risk index and a blend ratio.

FIG. 11 is a configuration example of an artifact correction section.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

Exemplary embodiments are described below. Note that the following exemplary embodiments do not in any way limit the scope of the content defined by the claims laid out herein. Note also that all of the elements described in the present embodiment should not necessarily be taken as essential elements.

1. Endoscope Apparatus

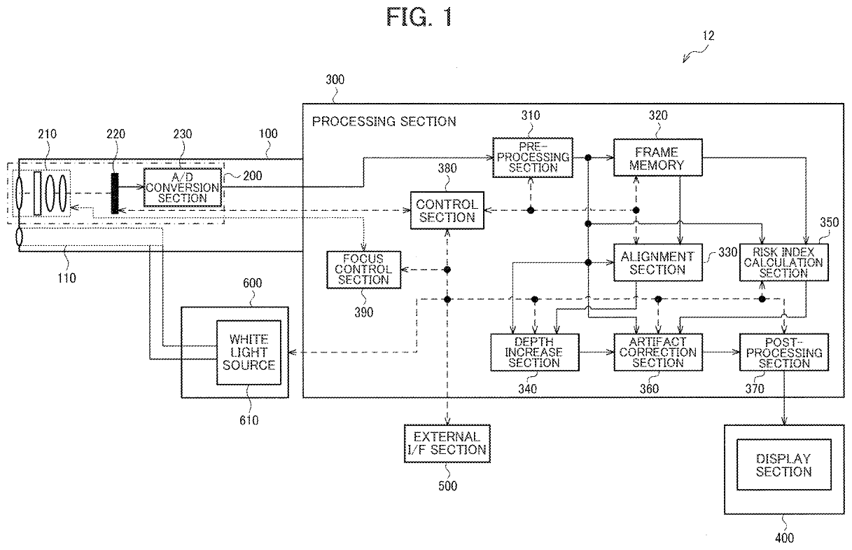

FIG. 1 is a configuration example of an endoscope apparatus 12. The endoscope apparatus 12 includes an insertion section 100, a processing section 300, a display section 400, an external OF section 500 and an illumination section 600. For example, the insertion section 100 is a scope, the display section 400 is a display device, the external OF section 500 is an interface, an operation section, or an operation device, and the illumination section 600 is an illumination device or a light source. The endoscope apparatus 12 includes a flexible scope used for a digestive tract or the like and a rigid scope used for a laparoscope or the like, for example.

The insertion section 100 is inserted into a living body. The insertion section 100 includes a light guide 110 and an imaging section 200.

The light guide 110 guides light emitted from the illumination section 600 to a distal end of the insertion section 100. The illumination section 600 includes a white light source 610, for example, and emits illumination light of white light. The white light source 610 is a light-emitting diode (LED) or a xenon lamp, for example. The illumination light is not limited to the white light, but illumination light of various bands used for the endoscope apparatus 12 may be employed.

The imaging section 200 forms an image of reflected light from a subject to capture an image of the subject. The imaging section 200 includes an objective optical system 210, an image sensor 220, and an A/D conversion section 230. The A/D conversion section 230 is an A/D conversion circuit, for example. The A/D conversion section 230 may be embedded in an image sensor.

The light guide 110 emits light to the subject. The objective optical system 210 forms an image from the reflected light from the subject as a subject image. A focus position of the objective optical system 210 is variable and is controlled by a focus control section 390 described later.

The image sensor 220 photoelectrically converts the subject image formed by the objective optical system 210 to capture the image. The A/D conversion section 230 converts analog signals sequentially output from the image sensor 220 into digital images to sequentially output to a preprocessing section 310. Specifically, the image sensor 220 shoots a video of the subject. The A/D conversion section 230 performs A/D conversion of images in respective frames of the video to output digital images to the preprocessing section 310. The preprocessing section 310 outputs a digital video.

The processing section 300 performs signal processing including image processing and control of the endoscope apparatus 12. The processing section 300 includes the preprocessing section 310, a frame memory 320, an alignment section 330, a depth increase section 340, a risk index calculation section 350, an artifact correction section 360, a postprocessing section 370, a control section 380, and the focus control section 390.

The preprocessing section 310 is a preprocessing circuit, and the postprocessing section 370 is a postprocessing circuit, for example. The frame memory 320 is a memory such as a random-access memory (RAM), for example. The alignment section 330 is an alignment circuit configured to calculate a motion vector. The depth increase section 340 is an image combination circuit, for example. The risk index calculation section 350 is a risk index calculation circuit, for example. The artifact correction section 360 is an image correction circuit, for example. The control section 380 is a control circuit or a controller, and the focus control section 390 is a focus control circuit or a focus controller, for example.

The preprocessing section 310 performs various image processing to the images sequentially output from the A/D conversion section 230, and sequentially outputs resultant images to the frame memory 320 and the depth increase section 340. The image processing includes a white balance process, an interpolation process, or the like, for example.

The frame memory 320 stores an M−1 image/M−1 images output from the preprocessing section 310, and outputs the image(s) to the depth increase section 340. M represents a number of images to be combined to generate a depth of field increased image, and is an integer of two or more.

The alignment section 330 performs alignment between images on a basis of the M−1 image(s) stored in the frame memory 320 and one image output from the preprocessing section 310. Specifically, the alignment section 330 sets one of the M images as a reference image for the alignment, and then aligns remaining image(s) with the reference image. The alignment may be implemented by a known block matching method, for example. The reference image used for the alignment is a latest image output from the preprocessing section 310, for example.

The depth increase section 340 combines the image output from the preprocessing section 310 and the M−1 image(s) output from the alignment section 330 after the alignment into a single depth of field increased image. That is, the depth increase section 340 combines the M images into the single depth of field increased image. The depth increase section 340 selects a best focused image of the M images for each local region in the depth of field increased image, extracts the local region of the selected image, and combines the extracted local regions into the depth of field increased image. The depth increase section 340 sequentially generates the depth of field increased images from the video shot by the imaging section 200 to produce a video including the depth of field increased images as frame images. The depth increase section 340 outputs the depth of field increased images to the artifact correction section 360.

The risk index calculation section 350 calculates a risk index indicating a risk of occurrence of the artifact in the depth of field increased image. The risk index is an index indicating a degree of the risk of occurrence of the artifact. The risk index is represented by numerical values, for example. An example of a case where a larger value of the risk index indicates a higher risk of occurrence of the artifact is described below. However, a relationship between the risk index and the risk indicated by the risk index can be implemented in various modified manners, such as a case where a smaller value of the risk index indicates a higher risk of occurrence of the artifact. The risk index used here is specifically an index indicating that the alignment by the alignment section 330 is inappropriate. That is, the higher risk indicated by the risk index specifically represents that the alignment is inappropriate and thus the risk of occurrence of the artifact is high. Details of the risk index calculation section 350 will be described later.

The artifact correction section 360 corrects the depth of field increased image output from the depth increase section 340 using the image output from the preprocessing section 310 on a basis of the risk index calculated by the risk index calculation section 350. Details of the artifact correction section 360 will be described later.

The postprocessing section 370 performs image processing, such as gamma processing, to the image corrected and output by the artifact correction section 360, and outputs a resultant image to the display section 400.

The control section 380 is bidirectionally connected to the image sensor 220, the preprocessing section 310, the frame memory 320, the alignment section 330, the depth increase section 340, the risk index calculation section 350, the artifact correction section 360, the postprocessing section 370, and the focus control section 390 to control these sections.

The focus control section 390 outputs a focus control signal for controlling a focus position to the objective optical system 210. The imaging section 200 captures images in M frames with different focus positions and the depth increase section 340 combines the M images into a single image. As a result, the depth of field increased image having an increased depth of field is obtained.

The display section 400 sequentially displays the depth of field increased images output from the depth increase section 340. That is, the display section 400 displays the video including the depth of field increased images as the frame images. However, the method according to the present embodiment is not limited to display video images, but the display section 400 may display still images. The display section 400 is a liquid crystal display or an electro-luminescence (EL) display, for example.

The external I/F section 500 is an interface used for input to the endoscope apparatus 12 by a user, for example. That is, the external I/F section 500 is, for example, an interface used for operation of the endoscope apparatus 12 or an interface used for performing operation setting of the endoscope apparatus 12. For example, the external I/F section 500 includes an adjustment button for adjusting a parameter for the image processing or the like.

As illustrated in FIG. 1, the endoscope apparatus 12 according to the present embodiment includes the imaging section 200 configured to acquire a plurality of images with different focus positions at different timings, the alignment section 330 configured to align the plurality of images with different focus positions, the depth increase section 340 configured to combine the plurality of images with different focus positions aligned by the alignment section 330 into a single depth of field increased image so as to increase the depth of field, the risk index calculation section 350 configured to calculate the risk index indicating the risk of occurrence of the artifact in the depth of field increased image, and the artifact correction section 360 configured to correct the depth of field increased image on a basis of the risk index.

The focus position used here is a position in focus on a subject side. That is, the focus position is a position of an in-focus plane or a position of an intersection of the in-focus plane and an optical axis. The focus position is represented by a distance from a reference position of the imaging section 200 to the position in focus on the subject side. The reference position of the imaging section 200 is, for example, a position of the image sensor 220, or a position of a distal end of an objective lens. The focus position is adjusted by moving a focus lens in the objective optical system 210. That is, the focus position and a position of the focus lens correspond to each other, and the focus position may be considered as the position of the focus lens.

Furthermore, the depth of field increased image is an image whose depth of field is increased compared with the depths of field of the captured images. Specifically, the depth of field increased image is an image whose depth of field is artificially increased on a basis of the plurality of images with different focus positions. For example, a best focused image of the M images is selected for each local region of the images, and the selected images for respective local regions are used to form the depth of field increased image. The local region is a pixel, for example. The M images to be combined into the depth of field increased image in a single frame are the images sequentially captured by the image sensor 220.

An EDOF technology for increasing the depth of field is widely known. In order to combine the images captured at different timings to generate the depth of field increased image, alignment between the images is important. Unfortunately, a conventional method disclosed in the Japanese Translation of PCT International Application Publication No. JP-T-2013-513318 does not consider whether the alignment can be appropriately performed. Thus, in a situation where the alignment is difficult, such as a case with a flat subject, or a case with an extremely large relative motion between the subject and the imaging section 200, increasing the depth may cause the artifact. In addition, during use of the endoscope apparatus, there may be cases where water is supplied or smoke is emitted by use of an electrosurgical knife. In such cases, the alignment with high accuracy is difficult, which may cause the artifact in the depth of field increased image.

On the other hand, the endoscope apparatus 12 according to the present embodiment calculates the risk index indicating the risk of occurrence of the artifact caused by alignment, and corrects the depth of field increased image according to the risk index. As a result, it is possible to switch which to focus on, increase of the depth of field or suppression of occurrence of the artifact, according to the risk index. That is, the endoscope apparatus outputs images according to the situation so as to allow a user to perform appropriate observation or treatment.

The endoscope apparatus 12 according to the present embodiment may have a configuration described below. That is, the processing section 300 includes a memory configured to store information, and a processor configured to operate based on the information stored in the memory. The information includes a program and various data, for example. The processor performs a focus control process, an image acquisition process, and image processing. The focus control process is for controlling the focus position of the objective optical system configured to form the subject image on the image sensor. The image acquisition process is for acquiring the images captured by the image sensor. The image processing includes a process of aligning the plurality of images with different focus positions, a process of combining the plurality of images after alignment into the single depth of field increased image, a risk index calculation process, and a correction process of correcting the depth of field increased image on a basis of the risk index.

The processor may have functions of sections each implemented by individual hardware, or the functions of sections each implemented by integrated hardware, for example. For example, the processor may include hardware, and the hardware may include at least one of a circuit that processes a digital signal and a circuit that processes an analog signal. For example, the processor may include one or more circuit devices mounted on a circuit board, or one or more circuit elements. The circuit device is an integrated circuit (IC), for example. The circuit element is a resistor or a capacitor, for example. The processor may be a central processing unit (CPU), for example. However, the processor is not limited to the CPU, but various other processors such as a graphics processing unit (GPU) or a digital signal processor (DSP) may also be used. The processor may be a hardware circuit that includes an application specific integrated circuit (ASIC). The processor may include an amplifier circuit, a filter circuit, or the like that processes an analog signal. The memory may be a semiconductor memory such as a static random-access memory (SRAM) or a dynamic random-access memory (DRAM), or may be a register. The memory may be a magnetic storage device such as a hard disk drive (HDD), or may be an optical storage device such as an optical disc device. For example, the memory stores a computer-readable instruction, and the processor performs the instruction to implement the function of each section of the processing section 300 as a process. The instruction used here may be an instruction set that is included in a program, or may be an instruction that instructs the hardware circuit included in the processor to operate. The processing section 300 includes the alignment section 330, the depth increase section 340, the risk index calculation section 350, and the artifact correction section 360. Alternatively, the processing section 300 may also include the control section 380, the focus control section 390, the preprocessing section 310, and the postprocessing section 370.

Furthermore, the sections of the processing section 300 according to the present embodiment may be implemented as modules of a program operating on the processor. For example, the alignment section 330 is implemented as an alignment module, the depth increase section 340 as an image combination module, the risk index calculation section 350 as a risk index calculation module, and the artifact correction section 360 as an image correction module.

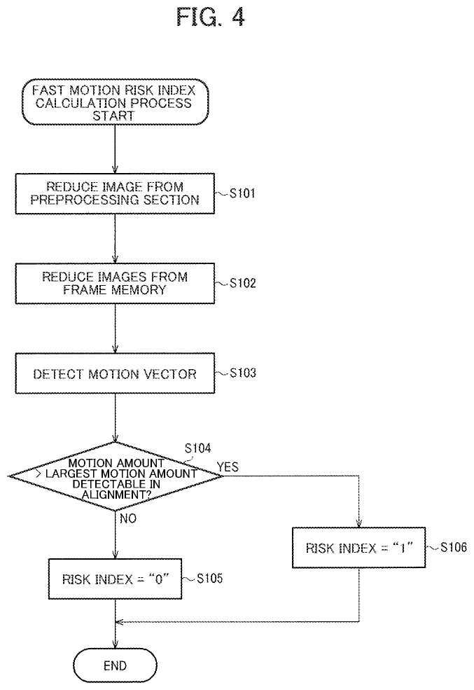

Furthermore, the program implementing the processes performed by the sections of the processing section 300 according to the present embodiment can be stored, for example, in a computer-readable medium such as an information storage device. The information storage device can be implemented by an optical disk, a memory card, an HDD, or a semiconductor memory, for example. The semiconductor memory is a read-only memory (ROM), for example. The processing section 300 performs various processes according to the present embodiment based on the program stored in the information storage device. That is, the information storage device stores the program causing a computer to function as the sections of the processing section 300. The computer is a device including an input device, a processing section, a storage section, and an output section. The program causes the computer to execute the processes of the sections of the processing section 300. Specifically, the program according to the present embodiment causes the computer to execute steps illustrated in FIGS. 4 and 6 to 8.

Furthermore, the method according to the present embodiment is applicable to other imaging devices for acquiring the depth of field increased image on a basis of the plurality of images with different focus positions. For example, the method according to the present embodiment is applicable to an imaging device such as a microscope.

2. Risk Index Calculation

Details of the risk index calculation section 350 are described below. A motion vector to be used for risk index calculation is described first, and then three specific risk indices are described. After that, some modifications in relation to the risk index calculation are described.

2.1 Motion Vector Calculation

The risk index calculation section 350 according to the present embodiment calculates the risk index on a basis of a motion vector between a plurality of images with different focus positions. The motion vector used here is information about a difference between a given subject position in the reference image for the alignment and a position corresponding to the given subject position in an image with a focus position different from the focus position of the reference image. The alignment section 330 performs the alignment on a basis of the motion vector. When the motion vector can not be detected accurately, combination of the plurality of images can not be performed in an appropriate positional relationship, and thus the risk of occurrence of the artifact in the depth of field increased image becomes high. That is, calculating the risk index on a basis of the motion vector allows accurate calculation of the risk of occurrence of the artifact caused by failure of the appropriate alignment.

Here, the alignment section 330 may detect the motion vector on a basis of the plurality of images with different focus positions and the risk index calculation section 350 may calculate the risk index on a basis of the motion vector detected by the alignment section 330. The alignment section 330 performs a process of detecting the motion vector on a basis of the plurality of images with different focus positions so as to perform the alignment. Various methods for detecting the motion vector are known, such as block matching, and these methods may be widely applicable to the present embodiment. Detecting a motion vector, which is to be used for the risk index calculation, by the alignment section 330 allows implementation of the endoscope apparatus 12 using an efficient configuration.

Furthermore, the alignment section 330 may detect the motion vector applicable to both the alignment and the risk index calculation. That is, the motion vector for the alignment may also be used for the risk index calculation. Alternatively, the alignment section 330 may separately detect a first motion vector for the alignment and a second motion vector for the risk index calculation.

Alternatively, the risk index calculation section 350 may detect the motion vector on a basis of the plurality of images with different focus positions and calculate the risk index on a basis of the detected motion vector. That is, separately from the detection of the motion vector for the alignment performed by the alignment section 330, the risk index calculation section 350 may detect the motion vector for the risk index calculation.

The alignment requires detection of a highly accurate motion vector in order to suppress the occurrence of the artifact. For example, the alignment section 330 detects the motion vector for each pixel. On the other hand, the risk index calculation only requires determination of whether the alignment has been performed with sufficient accuracy, and may not require the accuracy of the motion vector as high as that required by the alignment. Thus, detecting the motion vector by the risk index calculation section 350 allows implementation of a motion vector detection process according to the use.

2.2 Fast Motion Risk Index

The risk index calculation section 350 determines that the alignment with high accuracy can not be performed when a relative motion between the subject and the imaging section 200 is fast, and calculates the risk index indicating a high risk.

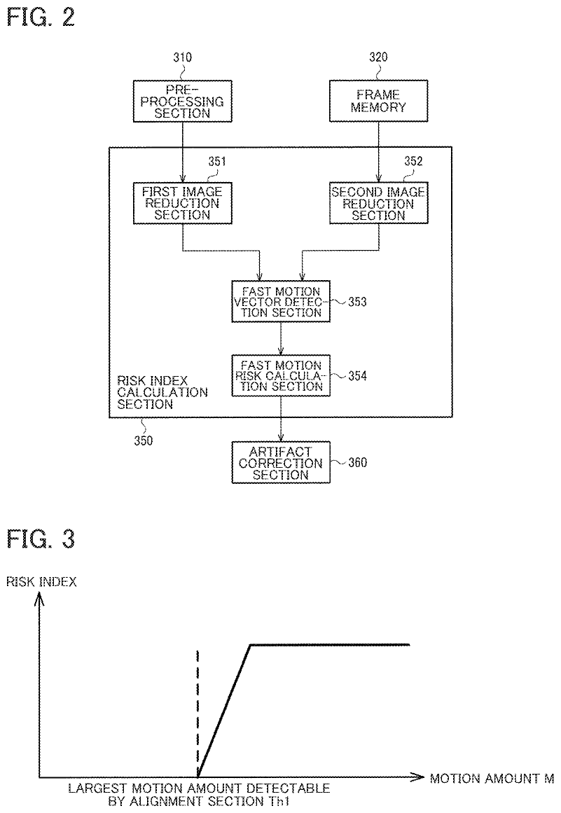

FIG. 2 is a configuration example of the risk index calculation section 350. The risk index calculation section 350 includes a first image reduction section 351, a second image reduction section 352, a fast motion vector detection section 353, and a fast motion risk index calculation section 354. The first image reduction section 351 reduces an image output from the preprocessing section 310. The reduced image is output to the fast motion vector detection section 353. The second image reduction section 352 reduces at least one image output from the frame memory 320. The reduced image is output to the fast motion vector detection section 353.

The fast motion vector detection section 353 detects the motion vector on a basis of the reduced images output from the first image reduction section 351 and the second image reduction section 352. Since the fast motion vector detection section 353 uses the reduced images to detect the motion vector, a more global motion is detected compared with the motion detected in detection of the motion vector by the alignment section 330 using the images before reduction. For example, when block sizes for the block matching are the same, the fast motion vector detection section 353 performs a matching process with a relatively large subject region as a target compared with a target of the alignment section 330. In addition, the matching process performed by moving the block by one pixel unit in the reduced image corresponds to the matching process performed by moving the block by a plurality of pixels unit in the image before reduction. That is, the fast motion vector detection section 353 performs the matching process by a larger unit compared with a unit used by the alignment section 330. The fast motion vector detection section 353 outputs information about the detected motion vector to the fast motion risk index calculation section 354.

Here, it is assumed that the alignment section 330 has an upper limit of a detectable motion amount. The upper limit of the detectable motion amount may be considered as an upper limit of the motion of the subject to be aligned. For example, in the block matching, the detectable motion amount is determined by a detection range in which a correlation (an evaluation value) between the blocks is obtained.

That is, the alignment section 330 is set with a maximum range in which the alignment is enabled. The risk index calculation section 350 calculates the risk index indicating a high risk when the motion amount indicated by the motion vector overwhelms the maximum range. Specifically, when the motion amount indicated by the motion vector overwhelms the maximum range, the risk index calculation section 350 calculates the risk index indicating a higher risk compared with a risk when the motion amount is equal to or smaller than the maximum range. However, when the motion vector is calculated using the reduced images as in the example illustrated in FIG. 2, a reduction ratio needs to be considered to compare “the maximum range in which the alignment is enabled” with “the motion amount indicated by the motion vector”. For example, when the first image reduction section 351 and the second image reduction section 352 reduce each side of the image by half, the maximum range in which the alignment is enabled (unit: pixel) and a magnitude of the motion vector (unit: pixel) are not directly compared. The motion amount is obtained by doubling the magnitude of the motion vector, for example. As a premise, the fast motion vector detection section 353 is set with the detection range of the motion vector such that a largest value of the detectable motion amount exceeds the maximum range in which the alignment is enabled.

The fast motion risk index calculation section 354 may calculate the fast motion risk index as binary information using “0” and “1”. For example, the fast motion risk index calculation section 354 calculates that the fast motion risk index is “1”, when the magnitude of the detected motion vector indicates a larger motion than a motion detectable by the alignment section 330. Otherwise, the fast motion risk index calculation section 354 calculates that the fast motion risk index is “0”. As described above, the fast motion risk index of “1” indicates a high risk of occurrence of the artifact, and the fast motion risk index of “0” indicates a low risk of occurrence of the artifact.

However, the fast motion risk index is not limited to the binary information, but may be calculated as multi-value information. FIG. 3 is a graph illustrating an example of calculating the fast motion risk index as the multi-value information. A horizontal axis in FIG. 3 represents a magnitude M of the motion amount corresponding to the motion vector detected by the fast motion vector detection section 353. A vertical axis in FIG. 3 represents a magnitude of the fast motion risk index. Th1 on the horizontal axis represents the largest value of the motion detectable by the alignment section 330, i.e., a value indicating the maximum range in which the alignment is enabled.

In the example in FIG. 3, the fast motion risk index calculation section 354 calculates that the fast motion risk index is “0”, when M≤Th1. The fast motion risk index calculation section 354 calculates that the fast motion risk index is a×(M−Th1), when M>Th1. a represents a coefficient of a>0, and a specific value of a may be implemented in various modified manners. In addition, in the example in FIG. 3, the risk index is set with a given largest value and the value of the risk index does not exceed the largest value. Calculating the fast motion risk index using a relationship illustrated in FIG. 3 allows the risk index calculation according to the motion amount that exceeds the largest motion amount detectable by the alignment section 330. FIG. 3 illustrates only an example of the calculation of the fast motion risk index. The fast motion risk index may be calculated using other relationships as long as the fast motion risk index becomes larger as M becomes larger.

As described above, according to the present embodiment, the risk index is calculated with the maximum range in which the alignment by the alignment section 330 is enabled as a criterion. When the maximum range in which the alignment is enabled is excessively small, the alignment is likely to be determined impossible, and an effect of increasing the depth of field by the EDOF is impaired. On the contrary, when the maximum range is excessively large, the alignment and the generation of the depth of field increased image are performed even with the fast motion, which causes the artifact. In addition, the excessively large maximum range extends the detection range, which increases a calculation load. That is, the maximum range in which the alignment is enabled is assumably set to a reasonable value in view of various conditions. Thus, setting the maximum range as the criterion also in the risk index calculation allows appropriate calculation of the fast motion risk index. Meanwhile, the fast motion vector detection section 353 according to the present embodiment needs to detect a larger motion compared with a motion detected by the alignment section 330. However, the motion vector for the calculation of the fast motion risk index is not required to be as accurate as the motion vector for the alignment. Thus, a processing load can be reduced by using the reduced images as a processing target, for example.

Meanwhile, one fast motion risk index can be calculated from one motion vector in both the cases where the fast motion risk index is calculated in binary and multiple values. According to the present embodiment, the fast motion vector detection section 353 may obtain one motion vector for the entire reduced image, and the fast motion risk index calculation section 354 may calculate one fast motion risk index from this motion vector. This corresponds to a case where the block size in the block matching is the same as a size of the reduced image. In such a case, one fast motion risk index is calculated for one depth of field increased image.

Alternatively, the fast motion vector detection section 353 may obtain a plurality of motion vectors for the reduced image, and the fast motion risk index calculation section 354 may calculate a plurality of fast motion risk indices respectively from the plurality of motion vectors to calculate the plurality of fast motion risk indices for each frame. In other words, a plurality of local regions are set in one depth of field increased image, and the fast motion risk index is calculated for each local region. Alternatively, the fast motion risk index calculation section 354 may calculate a plurality of fast motion risk indices and obtain a statistical value, such as an average value, of the plurality of fast motion risk indices to calculate one fast motion risk index for each frame.

FIG. 4 is a flowchart illustrating a fast motion risk index calculation process. When the process starts, the first image reduction section 351 reduces the image output from the preprocessing section 310 (S101). The second image reduction section 352 reduces the at least one image output from the frame memory 320 (S102). The fast motion vector detection section 353 detects the motion vector on a basis of the reduced image acquired in S101 and the reduced image acquired in S102 (S103). The fast motion risk index calculation section 354 determines whether the motion amount M indicated by the motion vector exceeds the largest motion amount Th1 detectable in the alignment (S104).

When M≤Th1 (No in S104), the fast motion risk index calculation section 354 calculates that the risk index is “0” (S105). When M>Th1 (Yes in S104), the fast motion risk index calculation section 354 calculates that the risk index is “1” or the value described referring to FIG. 3 (S106).

Furthermore, FIG. 2 illustrates the configuration in which the risk index calculation section 350 detects the motion vector to be used for the risk index calculation, however, the alignment section 330 may detect the motion vector as described above. In the latter case, the risk index calculation section 350 omits the first image reduction section 351, the second image reduction section 352, and the fast motion vector detection section 353 from the configuration illustrated in FIG. 2. The alignment section 330 performs the process of detecting the motion vector to be used for the alignment and processes corresponding to those performed by the first image reduction section 351, the second image reduction section 352, and the fast motion vector detection section 353. That is, the alignment section 330 reduces the image output from the preprocessing section 310, and the at least one image output from the frame memory 320, and detects the motion vector on a basis of the reduced images. The alignment section 330 outputs the motion vector detected on a basis of the reduced images to the fast motion risk index calculation section 354 of the risk index calculation section 350. For example, the alignment section 330 may perform a multiresolution analysis such as wavelet transformation to perform the alignment and output of the motion vector to the risk index calculation section 350.

Alternatively, the alignment section 330 may omit performing image reduction and perform the process of detecting the motion vector with a range exceeding the maximum range in which the alignment is enabled as the detection range. Then, the alignment section 330 performs the alignment when the detected motion vector is equal to or smaller than the maximum range. Further, the alignment section 330 outputs the information about the motion vector to the fast motion risk index calculation section 354 of the risk index calculation section 350.

2.3 Random Motion Risk Index

In an image captured using the endoscope apparatus 12, a motion of the subject is assumably caused by a motion of the insertion section 100. The motion of the insertion section 100 may include various motions such as a motion in an optical axis direction to change a distance from the subject, a translational motion, or a rotational motion. The subject in the image moves in a direction corresponding to the motion of the insertion section 100 in any case. That is, when a plurality of motion vectors are detected for the image, a given motion vector is considered to have a correlation with peripheral motion vectors to some extent.

Thus, when the plurality of motion vectors are detected for the image, the risk index calculation section 350 calculates the risk index on a basis of the correlation among the plurality of motion vectors. More specifically, the risk index calculation section 350 calculates the risk index such that a lower correlation among the plurality of motion vectors leads to a higher risk. The risk index is referred to as a random motion risk index herein. A case with the lower correlation among the motion vectors corresponds to a case where the accuracy of respective motion vectors is low. For example, it is considered that the correlation of the motion vectors decreases when visibility of the subject is significantly reduced by supplied water or emitted smoke.

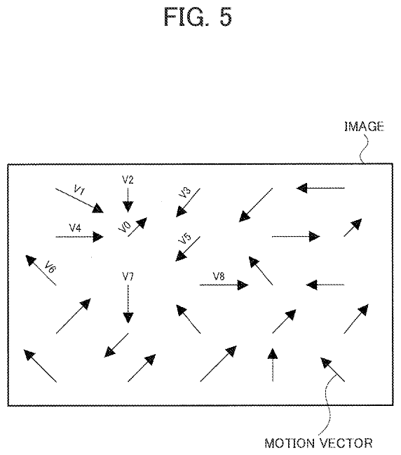

FIG. 5 is a diagram illustrating a random motion risk index calculation process. In an example illustrated in FIG. 5, a plurality of motion vectors are detected in an image. The risk index calculation section 350 sets a given motion vector as a target vector, and calculates that the random motion risk index is “1”, when the correlation between the target vector and the peripheral motion vectors is low. The risk index calculation section 350 calculates that the random motion risk index is “0”, when the correlation between the target vector and the peripheral motion vectors is high. The risk index calculation section 350 determines whether the correlation is high on a basis of a comparing process between a value indicating the correlation and a given threshold value, for example. In addition, the peripheral motion vectors may include four motion vectors on up and down, left and right sides of the target vector, eight motion vectors on a periphery of the target vector, or other combinations. For example, when a motion vector V0 is set as the target vector, the correlation with motion vectors V2, V4, V5, and V7 may be used or the correlation with motion vectors V1 to V8 may be used.

More specifically, the risk index calculation section 350 calculates that the random motion risk index is “1”, when a magnitude VD of difference vectors with respect to the peripheral motion vectors is larger than a threshold value Th2. When the plurality of motion vectors, such as four or eight motion vectors, are used as the peripheral motion vectors, a plurality of difference vectors are obtained with respect to the target vector. As for the magnitude of the difference vectors used for comparison with the threshold value, various statistical values, such as a sum, an average value, or a largest value, of magnitudes of the plurality of difference vectors may be used. This enables the risk index calculation when the alignment can not be performed due to a situation such as a water supply scene.

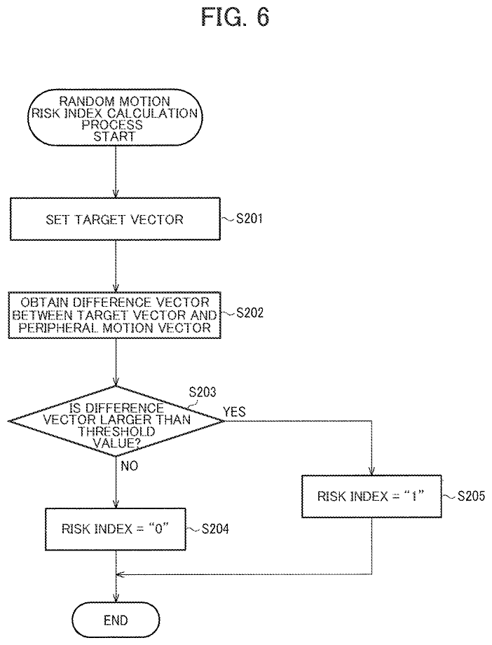

FIG. 6 is a flowchart illustrating the random motion risk index calculation process. When the process starts, the risk index calculation section 350 sets the target vector (S201), and obtains the difference vectors between the target vector and the peripheral motion vectors (S202). Then, the risk index calculation section 350 determines whether the magnitude VD of the difference vectors is larger than the given threshold value Th2 (S203).

When VD≤Th2 (No in S203), the risk index calculation section 350 calculates that the risk index is “0” (S204). When VD>Th2 (Yes in S203), the fast motion risk index calculation section 354 calculates that the risk index is “1” (S205).

As described above, the motion vector may be calculated by the risk index calculation section 350 or the alignment section 330. The water supply scene and a smoke emission scene are considered as situations causing the alignment to be difficult over the entire image. Accordingly, the risk index calculation section 350 calculates one random motion risk index for the entire image. However, the method according to the present embodiment is not limited to this, but the risk index calculation section 350 may divide the image into a plurality of regions and calculate the random motion risk index for each region. For example, the risk index calculation section 350 may set a plurality of target vectors to obtain a plurality of difference vectors. Then, the risk index calculation section 350 may calculate a random motion risk index for each target vector, or may calculate one random motion risk index on a basis of the plurality of target vectors.

In the water supply scene and the smoke emission scene, the alignment is very difficult, and an intermediate risk index between “0” and “1” is hardly presumed. Accordingly, FIG. 6 describes an example that the risk index calculation section 350 calculates the random motion risk index in binary. However, the risk index calculation section 350 may calculate the random motion risk index in multiple values.

2.4 Flat Region Risk Index and Periodic Structure Risk Index

The motion vector is a vector detected by calculating detection evaluation values each indicating the correlation between the plurality of images with different focus positions in the detection range, and detecting a detection evaluation value indicating a highest correlation. As for the detection evaluation value used herein, various evaluation values, such as a sum of absolute difference (SAD), a sum of squared difference (SSD), or a normalized cross correlation (NCC) may be used. With the SAD or SSD, a smaller detection evaluation value indicates a higher correlation between the images. With the NCC, a larger detection evaluation value, more particularly a value closer to one, indicates a higher correlation between the images.

When the detection evaluation values have one definite peak, reliability of the motion vector determined based on these detection evaluation values can be determined to be high. On the other hand, when the detection evaluation values have no definite peak, reliability of the motion vector determined based on these detection evaluation values is low. Specifically, a case with no definite peak corresponds to a case where the subject to be imaged is flat. Since the subject has little characteristic structure, the alignment is not performed appropriately, and the risk of occurrence of the artifact in the depth of field increased image is increased.

In addition, even if the detection evaluation values have the peak, when the peak is one of a plurality of peaks with the same degree, reliability of the motion vector determined based on these detection evaluation values is low. Specifically, a case with the plurality of peaks corresponds to a case where the subject has a periodic structure. An artificial object such as a treatment tool is sometimes imaged by the endoscope apparatus 12 and the treatment tool may include a base portion or the like having a periodic structure. In such a case, it is difficult to determine which one of the plurality of peaks is to be selected. Accordingly, the alignment is not appropriately performed and the risk of occurrence of the artifact in the depth of field increased image is increased.

Thus, the risk index calculation section 350 calculates the risk index on a basis of a difference between a detection evaluation value indicating a highest correlation and a detection evaluation value indicating a second highest correlation. Specifically, the risk index calculation section 350 calculates the risk index such that the risk becomes higher as the difference between the detection evaluation value indicating the highest correlation and the detection evaluation value indicating the second highest correlation is determined to be smaller. The risk index calculation section 350 determines that the difference is small when a difference indicated by difference information is small. Alternatively, the risk index calculation section 350 determines that the difference is small when a ratio indicated by ratio information is close to one. The difference information used here may be the difference itself, however, it is not limited to this and may include various information based on the difference. For example, the difference information may be information including a difference value applied with various processes, such as a normalization process or a correction process. The same applies to the ratio information. The ratio information may be the ratio itself, or information obtained based on the ratio.

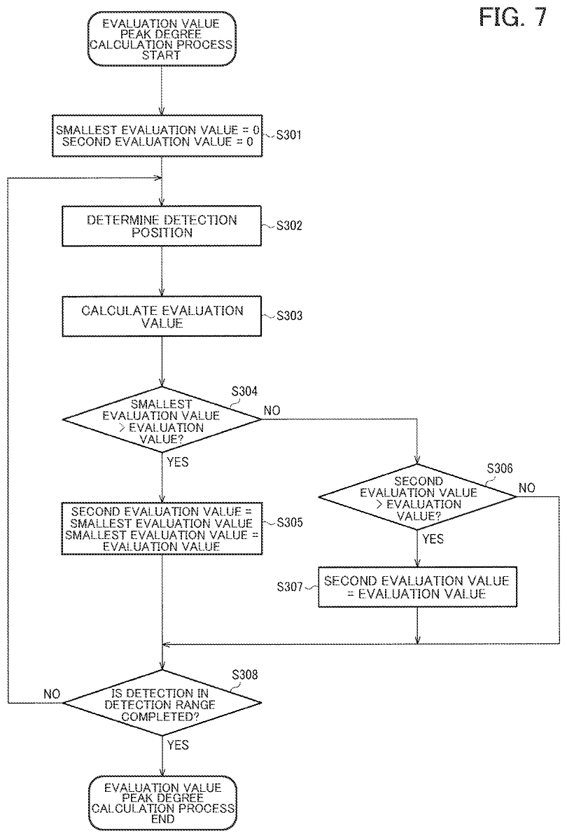

FIG. 7 is a flowchart illustrating a process of obtaining a peak value of the detection evaluation values. When the process starts, the alignment section 330 resets values of a smallest evaluation value and a second evaluation value to zero (S301). This is an example where a smaller detection evaluation value indicates a higher correlation, as with the SAD or SSD. That is, the smallest evaluation value is a detection evaluation value to be determined as having a highest correlation. The second evaluation value is a second smallest detection evaluation value to be determined as having a second highest correlation.

The alignment section 330 determines a given position in the detection range as a detection position (S302), and then calculates the detection evaluation value at the determined detection position (S303). When the detection evaluation value is the SAD, S303 is a process of calculating a sum of difference absolute values between an image region (a block) as a template and an image region at the detection position.

Next, the alignment section 330 compares the detection evaluation value calculated in S303 with the smallest evaluation value and determines whether the smallest evaluation value>the detection evaluation value (S304). When the detection evaluation value is smaller than the smallest evaluation value (Yes in S304), the alignment section 330 replaces the second evaluation value with the smallest evaluation value, and replaces the value of the smallest evaluation value with the value of the detection evaluation value calculated in S303 (S305).

When the detection evaluation value is equal to or larger than the smallest evaluation value (No in S304), the alignment section 330 determines whether the second evaluation value>the detection evaluation value (S306). When the detection evaluation value is smaller than the second evaluation value (Yes in S306), the alignment section 330 replaces the value of the second evaluation value with the value of the detection evaluation value calculated in S303 (S307). When the detection evaluation value is equal to or larger than the second evaluation value (No in S306), the alignment section 330 maintains the values of the smallest evaluation value and the second evaluation value.

The alignment section 330 determines whether the detection in the detection range is completed (S308). When the detection is not completed (No in S308), the alignment section 330 returns to S302 and continues the process. When the detection in the detection range is completed, the alignment section 330 terminates the process of obtaining the peak of the detection evaluation values. When obtaining the motion vector, the alignment section 330 stores the detection position corresponding to the smallest evaluation value, and performs the alignment with a vector connecting a reference position with the stored detection position as the motion vector.

In the example described above, the alignment section 330 performs the process of obtaining the peak value of the detection evaluation values. However, the process in FIG. 7 largely overlaps the process of obtaining the motion vector. That is, when the risk index calculation section 350 detects the motion vector, the risk index calculation section 350 may perform the process described referring to FIG. 7.

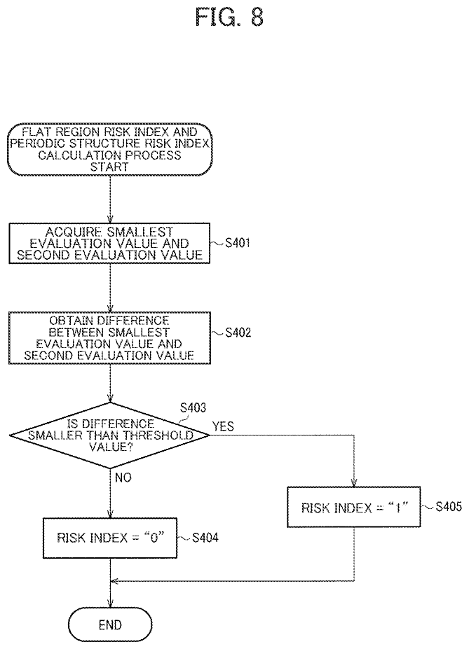

FIG. 8 is a flowchart illustrating a calculation process of a flat region risk index or a periodic structure risk index. When the process starts, the risk index calculation section 350 acquires the smallest evaluation value and the second evaluation value (S401), and obtains a difference between the smallest evaluation value and the second evaluation value (S402). Then, the risk index calculation section 350 determines whether the difference Dif is smaller than a given threshold value Th3 (S403). When Dif≥Th3 (No in S403), the risk index calculation section 350 calculates that the risk index is “0” (S404). When Dif<Th3 (Yes in S403), the risk index calculation section 350 calculates that the risk index is “1” (S405).

This enables calculation of the risk index with respect to a repeated pattern such as the base portion of the treatment tool and the flat subject.

As for the flat region risk index or the periodic structure risk index, a single value may be obtained for the entire image, or for each region. In the example in FIG. 8, the risk index calculation section 350 calculates the flat region risk index or the periodic structure risk index in binary. However, the risk index calculation section 350 may calculate the flat region risk index or the periodic structure risk index in multiple values.

2.5 Modifications of Risk Index Calculation

The above description is the example of using the detection evaluation value for detecting the motion vector to calculate the flat region risk index. However, the flat region risk index can be calculated on a basis of a determination result of whether the subject is flat, and thus may be calculated using something other than the detection evaluation value.

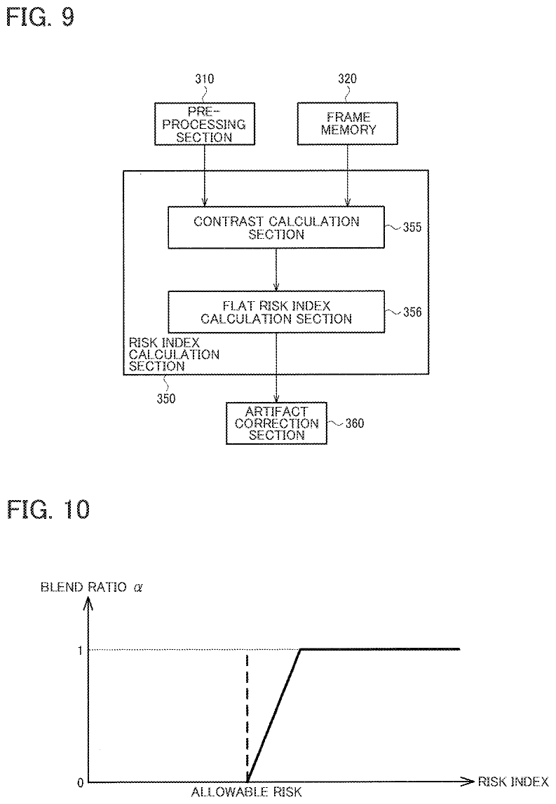

FIG. 9 is another configuration example of the risk index calculation section 350. The risk index calculation section 350 further includes a contrast calculation section 355 configured to calculate contrast values of the plurality of images with different focus positions. The contrast calculation section 355 calculates a contrast value of the image output from the preprocessing section 310, and a contrast value of the at least one image output from the frame memory 320. The contrast calculation section 355 outputs the calculated contrast values to a flat region risk index calculation section 356.

The flat region risk index calculation section 356 calculates the risk index indicating a high risk when a contrast value is smaller than a given threshold value. The contrast value used here may be any one of the contrast values obtained from the plurality of images with different focus positions, or a statistical value such as an average value or a smallest value. The contrast value is an output of a known bandpass filter, for example. The bandpass filter used here has frequency characteristics allowing extraction of typical structures imaged by the endoscope apparatus 12. The typical structures include a blood vessel structure, for example. There are various known methods for detecting the contrast value from the image, and these methods may be widely applicable to the present embodiment.

The flat region risk index calculation section 356 compares a contrast value Ct with a given threshold value Th4. The flat region risk index calculation section 356 calculates that the risk index is “1” when Ct<Th4, and is “0” when Ct≥Th4.

In the above description, it is assumed that the risk index calculation section 350 calculates the risk index with the entire depth of field increased image as a target. That is, in either cases where one risk index is calculated for the entire depth of field increased image and where the risk index is calculated for each local region in the depth of field increased image, it is assumed that the risk index corresponding to an arbitrary position in the depth of field increased image can be specified. This can balance increase of the depth and suppression of the artifact in a wide range of the image.

However, the risk index calculation section 350 may calculate the risk index with a partial region of the depth of field increased image as the target. In other words, a target region of the risk index calculation may be limited to part of the depth of field increased image.

For example, when a treatment tool such as an electrosurgical knife or a forceps is put out from a distal end of the insertion section 100 to perform treatment, the risk of occurrence of the artifact is high in a peripheral region of the treatment tool. For example, when the treatment tool is put in and out from the distal end of the insertion section 100, a position of the treatment tool changes in the image. Alternatively, when the insertion section 100 is moved with the treatment tool put out, the position of the treatment tool is fixed in the image, however, the subject corresponding to a background moves. In either case, the alignment is not appropriately performed due to a factor, for example, that part of the subject is blocked by the treatment tool and is prevented to be imaged, which may cause the artifact.

In such a case, the risk index calculation section 350 sets a partial region of a periphery of a region including an image of the treatment tool in the depth of field increased image as the target of the risk index calculation. This enables efficient risk index calculation targeting only an important region. A region of the image that is to include the image of the treatment tool can be determined based on a configuration of the insertion section 100 or the treatment tool. Thus, the partial region to be the target of the risk index calculation may be a predetermined region. Alternatively, since the treatment tool has chroma lower than that of a living body, the treatment tool can be detected by image processing. Accordingly, the processing section 300 may perform a process of detecting the region including the image of the treatment tool in the captured image, and set the detected region including the image of the treatment tool as the target region of the risk index calculation.

3. Artifact Correction

Next, the correction process of the depth of field increased image on a basis of the calculated risk index is described below. Specifically, the correction process includes blending or replacement between the depth of field increased image and an original image. The risk index described below may be one or a combination of two or more of the various risk indices described above. When two or more risk indices are input, for example, the artifact correction section 360 performs the correction process, described below, using a largest value of the risk indices, to focus on the suppression of the artifact. However, specific processing may be implemented in various modified manners, such as using an average value of the risk indices.

3.1 Blending Process

The artifact correction section 360 performs the correction process of blending at least one pixel or more in the depth of field increased image with any one of the plurality of images with different focus positions on a basis of the risk index. Blending two images as used herein corresponds to a process of obtaining a pixel value in a corrected image by weighting and adding a pixel value in one image to a pixel value in another image. Specifically, the artifact correction section 360 determines the pixel value of each pixel in the corrected image by Formula (1) below. α in Formula (1) below is a blend ratio determined on a basis of the risk index.

Corrected Image=(1.0−α)×depth of field increased image+α×original image (1)

FIG. 10 is a graph illustrating a relationship between the risk index and the blend ratio α. In FIG. 10, a horizontal axis represents a value of the risk index and a vertical axis represents the blend ratio α. The artifact correction section 360 is set with an allowable risk as an upper limit value of an allowable risk index. The allowable risk is an upper limit value of the risk index up to which the artifact is unlikely to occur and a problem is unlikely to occur if the depth of field increased image is displayed as it is. The allowable risk may be a fixed value. Alternatively, the allowable risk may be a dynamically set value based on a subject to be observed, a history of the risk indices previously calculated, or the like. As illustrated in FIG. 10, the blend ratio α is set to zero in a range where the calculated risk index is equal to or lower than the allowable risk. That is, the depth of field increased image is output as it is as the corrected image. When the calculated risk index is larger than the allowable risk, a lager risk index indicates a larger blend ratio α, so that a contribution degree of the original image to the corrected image increases. Note that, with Formula (1) described above, a largest value of the blend ratio α is one.

The original image used here is any one of the M images with different focus positions combined into the depth of field increased image. Considering that the depth of field increased image is an image generated by aligning the M−1 image(s) with respect to the reference image for the alignment, the original image to be used for blending is preferably the reference image for the alignment. The reference image for the alignment is the latest image output from the preprocessing section 310 as described above.

However, the reference image for the alignment may be an image output from the frame memory 320. The original image to be used for blending may also be an image other than the reference image for the alignment. For example, the artifact correction section 360 may set selection evaluation values respectively for a plurality of focus positions, and select an image captured at a focus position with a high selection evaluation value as the image to be used for blending. The selection evaluation value may be obtained from a size of a region determined to be in-focus, a number of selected times as the image to be used for blending, or the like.

As can be understood from Formula (1) described above, the correction process by blending well suits a case where the risk index is calculated in multiple values. With the risk index in multiple values, the blend ratio can be flexibly set in accordance with a value of the risk index. In addition, the correction process using Formula (1) described above well suits a case where the plurality of risk indices are calculated for the depth of field increased image. When the image includes a region having a high risk index and a region not having the high risk index, setting the blend ratio in accordance with the risk index using Formula (1) described above allows acquisition of a natural corrected image without a prominent boundary of the regions.

However, according to the present embodiment, the correction process by blending may be performed when the risk index is calculated in binary. For example, the artifact correction section 360 outputs the depth of field increased image as it is as the corrected image when the risk index is “0”, and outputs the corrected image including the depth of field increased image and the original image blended at a given blend ratio when the risk index is “1”.

Furthermore, according to the present embodiment, the correction process by blending may be performed when one risk index is calculated for the depth of field increased image. For example, the artifact correction section 360 outputs an image including the depth of field increased image and the original image blended at an uniform blend ratio over the entire image as the corrected image.

3.2 Replacement Process

The artifact correction section 360 may perform the correction process of replacing at least one pixel or more in the depth of field increased image with any one of the plurality of images with different focus positions on a basis of the risk index. The image to be used for replacement can be implemented in various modified manners as in a case of blending.

The correction process by replacement well suits a case where the risk index is calculated in binary. The artifact correction section 360 prioritizes the increase of the depth when the risk index is “0”, and sets a pixel value of the depth of field increased image as a pixel value of the corrected image. The artifact correction section 360 prioritizes the suppression of the artifact when the risk index is “1”, and replaces the depth of field increased image with the original image. The replacement corresponds to a process of setting the pixel value of the original image as the pixel value of the corrected image.

However, the correction process by replacement may be performed when the risk index is calculated in multiple values. For example, the artifact correction section 360 outputs the depth of field increased image as it is when the risk index is equal to or smaller than a given threshold value, and replaces the depth of field increased image with the original image when the risk index is larger than the given threshold value. The threshold value used here is the allowable risk illustrated in FIG. 10, for example.

In the case of the replacement, differently from the case of blending, an intermediate image between the depth of field increased image and the original image is not generated. In view of preventing the boundary of the regions from being prominent, the correction process by replacement better suits a case where one risk index is calculated for the depth of field increased image, compared with a case where a plurality of risk indices are calculated for the depth of field increased image. However, the correction process by replacement may be performed when the plurality of risk indices are calculated for the depth of field increased image.

Furthermore, when the risk indicated by the risk index is high over the entire image, partial output of the depth of field increased image is likely to make the artifact prominent, and thus it is preferable to output the original image as it is to prioritize the suppression of the artifact.

However, when the depth of field increased image is replaced with the original image, the corrected image does not include a contribution of the depth of field increased image, and thus the depth of field may be shallow and the subject may be blurred in the image. Accordingly, the artifact correction section 360 may further include a highlighting section 361 configured to perform a highlighting process of highlighting at least a region to be used to replace the depth of field increased image in any one of the plurality of images with different focus positions. The artifact correction section 360 (a replacement processing section 362) performs the correction process of replacing at least one pixel or more of the depth of field increased image with an image applied with the highlighting process based on the risk index.

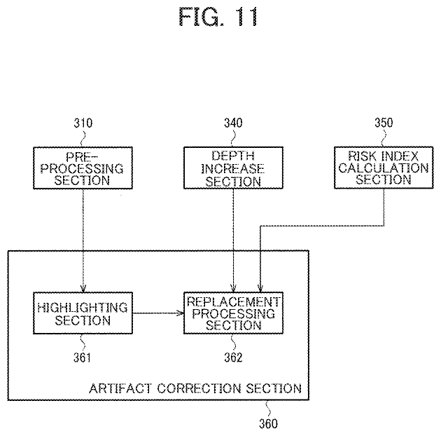

FIG. 11 is a configuration example of the artifact correction section 360. The artifact correction section 360 includes the highlighting section 361 and the replacement processing section 362. The highlighting section 361 acquires the image from the preprocessing section 310 as the original image for the replacement process, and performs the highlighting process. The highlighting process used here is a process of enhancing a structure in the original image, and is specifically an edge enhancement process. The replacement processing section 362 receives the original image applied with the highlighting process from the highlighting section 361, the depth of field increased image from the depth increase section 340, and the risk index from the risk index calculation section 350. The replacement processing section 362 performs the process of replacing the depth of field increased image with the original image applied with the highlighting process based on the risk index. This can enhance the visibility of the structure of the subject even when the depth of field is not increased due to the replacement.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

What is claimed is:

1. An endoscope apparatus comprising:
an imaging device that acquires a plurality of images with different focus positions at different timings; and
a processor including hardware,
the processor being configured to
align the plurality of images with different focus positions,
combine the plurality of images with different focus positions that have been aligned into a single depth of field increased image to increase a depth of field,
obtain a risk index indicating a degree of a risk of occurrence of artifact in the depth of field increased image, and
correct the depth of field increased image on a basis of the risk index.

2. The endoscope apparatus as defined in claim 1,
the processor
obtaining the risk index on a basis of a motion vector between the plurality of images with different focus positions.

3. The endoscope apparatus as defined in claim 2,
the processor
detecting the motion vector on a basis of the plurality of images with different focus positions, and obtaining the risk index on a basis of the motion vector thus detected.

4. The endoscope apparatus as defined in claim 2,
the processor
detecting the motion vector on a basis of the plurality of images with different focus positions, and obtaining the risk index on a basis of the motion vector thus detected.

5. The endoscope apparatus as defined in claim 2,
alignment of the plurality of images with different focus positions being performed with a maximum range in which the alignment is enabled, and
the processor
calculating the risk index indicating a high risk when a motion amount indicated by the motion vector overwhelms the maximum range.

6. The endoscope apparatus as defined in claim 2,
a plurality of motion vectors being detected for at least one of the plurality of images, and
the processor
calculating the risk index on a basis of a correlation between the plurality of motion vectors.

7. The endoscope apparatus as defined in claim 2,
the motion vector being detected by obtaining detection evaluation values each indicating a correlation between the plurality of images with different focus positions and detecting a detection evaluation value indicating a highest correlation in a detection range, and
the processor
calculating the risk index on a basis of any one of difference information and ratio information between the detection evaluation value indicating the highest correlation and a detection evaluation value indicating a second highest correlation.

8. The endoscope apparatus as defined in claim 1,
the processor
calculating contrast values of the plurality of images with different focus positions, and calculating the risk index indicating a high risk when a contrast value is smaller than a given threshold value.

9. The endoscope apparatus as defined in claim 1,
the processor
obtaining the risk index with a partial region of the depth of field increased image as a target.

10. The endoscope apparatus as defined in claim 1,
the processor
obtaining the risk index with an entire region of the depth of field increased image as a target.

11. The endoscope apparatus as defined in claim 1,
the processor
performing a correction process of replacing at least one pixel or more in the depth of field increased image with any one of the plurality of images with different focus positions on a basis of the risk index.

12. The endoscope apparatus as defined in claim 1,
the processor
performing a correction process of blending at least one pixel or more in the depth of field increased image with any one of the plurality of images with different focus positions on a basis of the risk index.

13. The endoscope apparatus as defined in claim 11,
the processor
performing a highlighting process on at least a region of any one of the plurality of images with different focus positions to be used to replace the depth of field increased image, and performing a correction process of replacing at least one pixel or more in the depth of field increased image with the one of the plurality of images applied with the highlighting process on a basis of the risk index.

14. An operating method of an endoscope apparatus comprising:
acquiring a plurality of images with different focus positions at different timings;
aligning the plurality of images with different focus positions;
combining the plurality of images with different focus positions that have been aligned into a single depth of field increased image to increase a depth of field;
obtaining a risk index indicating a degree of a risk of occurrence of artifact in the depth of field increased image, and
correcting the depth of field increased image on a basis of the risk index.

15. A non-transitory information storage medium storing a program,
the program causing a computer to perform steps of:
acquiring a plurality of images with different focus positions at different timings;
aligning the plurality of images with different focus positions;
combining the plurality of images with different focus positions that have been aligned into a single depth of field increased image to increase a depth of field;
obtaining a risk index indicating a degree of a risk of occurrence of artifact in the depth of field increased image, and
correcting the depth of field increased image on a basis of the risk index.
